(19) United States
(12) Patent Application Publication
(10) Pub. No.: US 2009/0253621 A1
(43) Pub. Date: Oct. 8, 2009

(54) ARABINOGALACTAN PROTEIN HAVING THE PROPERTY OF ABSORBING FATS AND METHOD FOR OBTAINING THIS ARABINOGALACTAN PROTEIN

(75) Inventor: Mohamed Elaghdef Malainine, Marrakech (MA)

Correspondence Address: The Marbury Law Group, PLLC, 11800 SUNRISE VALLEY DRIVE, SUITE 1000, RESTON, VA, 20191, US

(73) Assignee: UNIVERSITE CADI AYYAD

(21) Appl. No.: 12/097,363

(22) PCT Filed: Dec. 13, 2006
(86) PCT No.: PCT/MA06/00005

§ 371 Date: Jun. 13, 2008

(30) Foreign Application Priority Data
Dec. 15, 2005 (MA) 28668

(51) Int. Cl. A61K 38/00

(52) U.S. Cl. 514/12

(57) ABSTRACT

The invention relates, firstly, to a method for eliminating ingested dietary fats before they are assimilated by the body, using an AGP extracted from cactus mucilage. Said AGP can be in any galenic or commercial form which is solid or dissolved in water. The invention also discloses the method of extracting this AGP from the mucilage of a cactus of the Platyopuntia genus, by means of a process which makes it possible to preserve the polysaccharide-protein association, which association is responsible for the observed activity.

Acronym: AGP(s)=Arabinogalactan proteins)

AG(s)=Arabinogalactan(s)

### TECHNICAL FIELD OF THE INVENTION

The present invention relates to methods for controlling weight and reducing cholesterol without the need of restriction on food caloric intake

### STATE OF THE PRIOR ART

In the field of methods for controlling weight and reducing cholesterol without any restriction on food caloric intake, several techniques are known:
Inhibition of lipases, [T. Kazuyo & H. Tetsuya patent WO2005099735]
Blocking fatty acid receptors [Xenical de Roche, marketed as Oristat]
Physical complexation of the lipids, etc . . .

In the first two techniques, the question is to neutralize physiological processes, by using active drugs on the organism.

In the last technique, the question is to promote physical interaction between the lipids and a food compound which does not have any other particular physiological interactions on the organism.

As such, only two products are recognized as effective materials: chitosan [Furda. U.S. Pat. No. 4,223,023 (1980)] and nopal powder [D'Huart & Dallas, French Patent No. FR2823423 (2002)].

From the point of view of effectiveness, the first, even added with fibers, has limited binding capacity under physiological conditions. Further, because of the change in pH in the intestines, there occurs a lipid salting-out phenomenon which releases them from binding to chitosan. The result is that lipids may be assimilated by the organism.

As for nopal powder, it is a heterogeneous material of natural origin therefore with variable composition over time, but which has demonstrated a capacity of binding fats which is equivalent or even larger than that of chitosan.

In fact, these are leaves (also called cladodes), of certain edible cactaceae of the Platyopuntia type, and especially of the species Opuntia ficus indica (commonly called nopal), which are consumed within the scope of food diets for treating obesity, hyperlipidemia, and diabetes. A French patent on the use of cladodes in a preparation having the property of binding fats exists. [D'Huart & Dallas, French Patent No. FR2823423 (2002)].

In this patent, the whole dried and milled plant is used. The active ingredient responsible for the binding is not disclosed.

The problem is to find an active ingredient which is:
capable of better absorbing, binding and removing fats,
whereof this capability is maintained in a physiological medium,
having no potentially dangerous physiological interaction on the organism,
and which is stable over time.

AGPs are very wide spread in the vegetable world. This association of polysaccharides and proteins seems to have important physiological functions for plants. The osidic part (AG) also has a great industrial importance, whence a scientific interest marked by the number of publications and patents which relate to them. The structure of AGs was extensively studied. See for example E. G., Timell, Adv. Carbohydrate Chem., Vol. 20, pp. 409483 (1965).

Arabinogalactans are polysaccharides, the backbone of which consists of chains of beta-(1,3)-galactan, densely connected up by side chains consisting of arabinose, galactose units and often other minor residues.

One of the most ancient sources of AGs is acacia gum, an exsudate of Acacia Senegal. A more recent source of relatively pure AGs is the wood extract from Larex Occidentalis. These AGs are used in the food and agriculture industry and the cosmetic industry as a texture agent, a gelling agent and as an emulsifier facilitating water-oil mixing.

No claim or any prior publication directly relates an AGP to a fat absorption and binding property.

This is the main object of this invention.

### SUMMARY OF THE INVENTION

Firstly, this is a method for eliminating ingested dietary fats before they are assimilated by the body, using an AGP extracted from cactus mucilage. Said AGP may be in any galenic or commercial form, which is solid or dissolved in water.

This is also a method for extracting this AGP from the mucilage of a cactus of the platyopuntia genus, by means of a process which makes it possible to preserve the polysaccharide-protein association, which association is responsible for the observed activity. OBJECT OF THE INVENTION

The invention is directed to proposing a method based on a product, and to a method for obtaining such a product, having the property of binding fats. It is directed to proposing a product which may be ingested without any risk (i.e. not exhibiting any noxiousness) and having the property of binding fats in vivo in order to prevent the fats from being digested.

The invention first aims at a method for absorbing, binding and then removing ingested dietary fats before their assimilation by the organism by using an AGP extracted from cactus mucilage.

Commercial AGs all have a good emulsifying capability. It is normal to expect that this capability improves transport of dietary fats and their subsequent assimilation by the organism which ingests them.

One skilled in the art expects that AGP extracted from cactus mucilage behaves in a similar way, considering the structure of the polysaccharide which makes it up.

But if the AGP described in this invention is used, after it being mixed with a water-oil mixture, (see: binding test in the Examples), a temporary emulsion is formed which produces a gel which traps the whole fatty phase. This effect makes fats unavailable for assimilation by the organism which ingests them.

Because of its solubility in water, the AGP extracted from the cactus, object of this invention, may appear in any galenic or commercial form, which is solid or dissolved in water. It may be packaged alone, as tablets, gelatin capsules, flexible or rigid capsules, or quite simply powder to be dissolved in a drink (water or another fizzy drink, fruit juice, etc.) or to be sprinkled on a foodstuff. It is then preferably ingested during a meal.

The invention is therefore extended to any preparation containing AGP extracted from the mucilage of a cactus, having the property of binding ingested dietary fats, so as to minimize their assimilation.

It is also the method for extracting this AGP from the mucilage of a cactus of the platyopuntia genus, by means of a process by which the polysaccharide-protein association, which association is responsible of the observed activity, may be preserved.

This method of extraction without any solvent, without any added foreign material, represents a real guarantee on the innocuousness of the AGP extracted from the cactus mucilage.

In a first phase, the problem is to isolate the (very soluble) AGP from the other constituents of the cactus without deteriorating it. This effect is advantageously achieved according to the invention, by macerating purified water under an inert atmosphere, either with cactus pads, cut beforehand into pieces (alternative 1), or with powder from dried cactus pads (alternative 2).

Maceration should be carried out quite quickly, but at a low temperature. Maceration is immediately followed by a solid-liquid separation. The separation is advantageously performed by centrifugation and filtration.

The solution of obtained AGPs is then purified. This purification is advantageously achieved according to the invention, by submitting it to dialysis against distilled water, in order to remove the salts and the molecules of low molecular weight.

The purified AGP solution is then concentrated. Concentration is advantageously achieved according to the invention in an enclosure partly in vacua and at a low temperature.

The AGP concentrated solution is then dried. Removal of water may be accomplished in two advantageous ways according to the invention, either by freeze-drying or by atomization.

In the exemplary embodiments of the invention, several possibilities are given as an indication and are not limiting.

### EXAMPLES

1. Binding Test

The AGP produced according to the invention is compared with commercial products having the property of binding fats.

Operating Procedure of the Binding Test

Material:

Oven adjusted to 37° C.

Scales with an accuracy of 0.1 mg

250 mL beaker

Plugged 85 mL glass tubes to be centrifuged (dimensions 44×98 mm)

Micropipettes of 100 μL and 1,000 μL

Centrifuge adjusted to 2,000 rpm (670 g)

Reagents:

0.1N hydrochloric acid

| | | |
|---|---|---|
| Buffer: tris(hydroxymethyl)aminomethane acid | 120 | g |
| 35% HCl | 49 | ml |
| Water | 200 | ml |

Operations:

Five test tubes each containing 7.5 mL of 0.1N HCl and 6 g (mass M) of sunflower oil, are prepared. 100 mg (mass M1) of the preparation to be tested (cactus powder of the Neopuntia® type, chitosan of the Absorbitol® type, commercial AG of the Larex UF® type or acacia gum, or AGP prepared according to the invention) are placed in each tube.

The obtained solutions are vigorously mixed, by stirring the tube by hand for 30 s, and then placed in an oven at 37° C. for 2 hour incubation. 0.5 mL of a pH 7.3 buffer solution is then added into each of the tubes. After fresh manual stirring, the tubes are put back into the oven at 37° C. for 3 hours. They are then centrifuged for 5 minutes at 2,000 rpm. The supernatant fat is then picked Lip and weighed (mass M2), and the ratio of the mass of fat bound by the preparation (M-M2) over the applied preparation mass (M1), the so-called binding ratio, is calculated.

For each tested preparation, the test is repeated three times.

Results

| | Registered trade mark | Binding |
|---|---|---|
| Preparation | or trade name | ratio* |
| Cactus powder | Neopuntia ® | 12 ± 2 |
| Chitosan | Absorbitol ® | 11 ± 2 |
| Arabinogalactan of larex | Larex UF ® | 0 |
| occidentalis | | |
| Acacia gum from | Acacia gum | 4 ± 2 |
| acacia senegal | | |
| AGP according to the invention, | — | 31 ± 2 |
| alternative 1 | | |
| AGP according to the invention, | — | 28 ± 2 |
| alternative 2 | | |

*The binding ratio is defined by: the bound mass of oil (M-M2) divided by the mass of the preparation used (M1), R = (M-M2)/M1.

It is clear that the AGP prepared according to the invention not only surpasses all the fat binding agents known on the market (triple capacity), but also widely differs from commercial arabinogalactans which do not generally provide good binding ratios.

2. Example of a Method for Extracting AGP

The extraction method disclosed by this invention aims at preparing an arabinogalactan protein with a high molecular weight without causing failure of the bonds between the polysaccharide fragment and the protein fragment.

This is also a clean method, without any solvent, without any added foreign material, which represents a real guarantee of innocuousness of AGP extracted from cactus mucilage.

Equipment:

A lift truck with incorporated scales (sensitivity: 1 kg).

A stirred double jacket tank.

A centrifugal decanter NX 314, adjusted to 500 g.

A centrifuge-skimmer SC35, adjusted to 20,000 g.

A filter press ORION with 20 plates (40×40 cm).

Filtering plates of the KS80 type.

A falling film evaporator ADF.

An atomizing tower APV PSD 52.

A laboratory freeze-drier.

A membrane pump.

Flexible hoses.

### Operating Procedure, Alternative 1

100 kg of water purified by reverse osmosis are placed in the stirred tank, and nitrogen is bubbled in the water. Heating of the tank is then initiated and adjusted to 50° C. Once this temperature is attained, 100 kg of cut-out cactus pads of the opuntia ficus indica type are added. Stirring is maintained for 30 minutes, and then the contents of the tank are transferred via the membrane pump to the centrifugal decanter with a flow rate of 1,000 L/hr. The recovered liquid is transferred to clarification in the centrifuge-skimmer operating at 500 L/hr. The transparent juice is transferred though the filter press, on KS80 plates under a pressure of 2 bars. The bright juice obtained is then dialyzed against purified water, on membranes with a 10,000 Dalton cutoff, and then concentrated in the falling film evaporator ADF for 90 minutes. 1 L of concentrate is sampled in order to transfer it to freeze-drying. The remainder is transferred onto the atomizing tower operating at a hot air temperature of 190° C. In both cases (freeze-drying or atomization), the obtained AGP powder is of a pale yellow color.

### Operating Procedure, Alternative 2

This time, 300 kg of purified water are placed in the tank and 10 nitrogen is bubbled. Heating is initiated and one waits until the temperature reaches 50° C. With stirring, 20 kg of nopal powder with a particle size between 150 and 300 μm are added, the remainder of the procedure is unchanged.

### Characterization of AGP

In order to characterize the AGP extracted according to both procedures, we determined the composition of the polysaccharide (arabinose, galactose, xylose, rhamnose and uronic acids) and the percentage of protein in the AGP.

| | Alternative 1 | Alternative 2 |
|---|---|---|
| Arabinose | 40.8% | 31.3% |
| Galactose | 40.4% | 34.9% |
| Xylose | 7.2% | 13.0% |
| Rhamnose | 2.3% | 3.4% |
| Uronic acids | 2.3% | 3.4% |
| Proteins | 7.0% | 14.1% |

1. A method for binding dietary fats contained in a meal in order to prevent their assimilation by a hot-blooded organism, wherein the agent used for achieving binding is an arabinogalactan protein.

2. The method according to claim 1, wherein the arabinogalactan protein is extracted from an edible cactus of the platyopuntia genus.

3. The method according to claim 1, wherein the arabinogalactan protein includes a ratio of arabinose to galactose comprising between about 0.3 and about 1.6.

4. The method according to claim 1, wherein the protein weight in the arabinogalactan protein comprises between about 7% and about 14%.

5. A method for extracting an arabinogalactan protein from the mucilage of an edible cactus, comprising:
macerating cladodes of said cactus in water;
separating the insoluble materials;
purifying the resulting solution of arabinogalactan protein by osmosis; and
concentrating the solution and removing water;
wherein the maceration is carried out in an inert atmosphere.

6. The method according to claim 5, wherein the temperature of the maceration comprises between about 15 and about 50° C.

7. The method according to claim 5, wherein separation is accomplished by centrifugation.

8. The method according to claim 5, wherein purification is accomplished by dialysis against pure water in suitable membranes.

9. The method according to claim 5, wherein concentrating is accomplished in vacuo at a temperature comprising between about 30 and about 45° C.

10. The method according to claim 5, wherein water removal is accomplished by freeze-drying.

11. The method according to claim 5, wherein water removal is accomplished by atomization.
